(19) United States
(12) Patent Application Publication
(10) Pub. No.: US 2018/0240561 A1
(43) Pub. Date: Aug. 23, 2018

(54) DIRECT SYNTHESIS OF RADIOACTIVE NANOPARTICLES INVOLVING NEUTRONS

(71) Applicants: Carlos Henry Castano Giraldo, Rolla, MO (US); Maria Camila Garcia Toro, Rolla, MO (US); Brian Michael Mills, Rolla, MO (US)

(72) Inventors: Carlos Henry Castano Giraldo, Rolla, MO (US); Maria Camila Garcia Toro, Rolla, MO (US); Brian Michael Mills, Rolla, MO (US)

(73) Assignee: The Curators of the University of Missouri, Columbia, MO (US)

(21) Appl. No.: 15/892,520

(22) Filed: Feb. 9, 2018

Related U.S. Application Data
(60) Provisional application No. 62/460,927, filed on Feb. 20, 2017.

(51) Int. Cl. G21G 1/06; G21G 1/12; B82Y 5/00

(52) U.S. Cl. CPC G21G 1/06; G21G 2001/0094; B82Y 5/00; G21G 1/12

(57) ABSTRACT

A method to synthesize radioactive nanoparticles includes the production of metallic and multimetallic nanoparticles in a single step by providing an aqueous solution of the metal precursor, and irradiating the aqueous solution thereby producing nanoparticles. The obtained nanoparticles include one or more radioactive isotopes of gold, such as ¹⁹⁸Au and ¹⁹⁹Au as well as radioisotopes of silver when the obtained nanoparticles are bimetallic. The aqueous solution is irradiated in a radiation field that includes neutrons and gamma rays. The radiation field may be provided by a nuclear reactor. The aqueous solution may include silver, and bimetallic nanoparticles may be produced. Duration of the irradiation time is selected to control the particle size distribution of the produced nanoparticles. The bimetallic nanoparticles can include core-shell nanoparticles and alloyed nanoparticles.

### CROSS-REFERENCE TO RELATED APPLICATION

This application claims the benefit of priority of U.S. provisional patent application No. 62/460,927, titled “DIRECT SYNTHESIS OF RADIOACTIVE NANOPARTICLES INVOLVING NEUTRONS,” filed on Feb. 20, 2017, which is incorporated herein in its entirety by this reference.

### GOVERNMENT RIGHTS

This invention was made with government support under contract No. NRC-HQ-12-G-38-0075 awarded by the Nuclear Regulatory Commission. The government has certain rights in the invention.

### TECHNICAL FIELD

The present disclosure relates to producing radioactive nanoparticles using a direct synthesis method. More particularly, the present disclosure relates to producing nanoparticles that include radioactive gold nuclei, and bimetallic nanoparticles.

### BACKGROUND

Cancer and heart diseases are the principal causes of death around the world. The American Cancer Society, with the help of the National Cancer Institute, the National Program of Cancer Registries, the National Center for Health Statistics and the North American Association of Central Cancer Registries, have projected that a total of 1,658,370 new cancer cases and 589,430 cancer deaths will have occurred in the United States during 2015. Accordingly, billions of dollars are being invested in research to increase the knowledge about causes, biology, new drugs and development of newer and more effective therapies to reduce the death of cancer patients.

Some disadvantages in the current cancer therapies include the inability to bypass biological barriers, poor delivery and inadequate distribution of drugs inside the body, and difficult detection by imaging. The application of nanotechnology in cancer treatment is helping overcome these limitations, giving the patients more possibilities to defeat the disease and extend life expectancy. Nanotechnology usage is the focus of exhaustive investigation in imaging and treatment.

Gold nanoparticles (AuNPs) have shown good results in cancer diagnosis and treatment due to their high stability, low reactivity, low toxicity levels for the human body, and easy functionalization process. AuNPs have been used to enhance imaging, photothermal therapy, chemotherapy, and radiation therapy by increasing the absorption or scattering of radiation. The newest research in cancer treatment using gold nanoparticles includes ¹⁹⁸Au and ¹⁹⁹Au radioactive isotopes for locally irradiating and killing tumor cells. The properties of these isotopes (¹⁹⁸Au half-life=2.7 days, β_{avg}=312 keV, βₘₐₓ=961 keV, and γ=412 keV; ¹⁹⁹Au half-life=3.1 days, β_{avg}=86 keV, βₘₐₓ=453 keV, and γ=159 keV) allow easy manipulation of the nanoparticles during transport and chemical processing before clinical applications.

The principal advantage of using radioactive AuNPs is the reduction of the chemotherapy time, dosage and side effects. Some studies have demonstrated that it is possible to kill cancer cells in tumors that have radioactive AuNPs using x-ray beam radiation instead of gamma irradiation.

Additionally, the development of nanostructures of multiple metals and alloyed metal represents a way to create new materials that show different structural, chemical, and physical properties that improve the behavior of the bulk material. Bimetallic nanoparticles (BMNPs) have shown improved performance due to their enhanced catalytic activity and selectivity in comparison with nanoparticles made using only one metal. These properties can be tuned modifying the size, composition, and structure of the nanoparticles. Bimetallic nanoparticles have applications ranging from heat transfer, chemical processes, electrochemical processes, electronic technology, aerospatial industry, and others. Currently, bimetallic nanoparticles play an important role in medicine, specifically in cancer therapy due to the advantages obtained from the different elements present in the nanostructure that improve their behavior for imaging, drug delivery and treatment.

Different methods to synthesize BMNPs are available including chemical processes (such as sol-gel, impregnation methods, liquid reduction methods, and others), biological processes, physical processes, and radiochemical reduction processes.

According to previous research, the size distribution of nanoparticles used in cancer treatment is a key element to improve tumor retention, interstitial interaction inside of the body, and the cancer cell killing process. Those studies have demonstrated that particles with diameters between 1 nanometer (nm) and 100 nm are smaller than the size of the pores in the typical tumor vasculature allowing the AuNPs to access the cells in the tumor. Nanoparticles with approximately 50 nm of diameter exhibited greater sensitization and higher cell uptake compared with other sizes.

Many studies have been published about the different ways to produce radioactive nanoparticles. Most of them require two different steps: first the nanoparticles are synthesized by chemical or physical processes, and then, as a second step, the obtained nanoparticles are activated with a neutron source. This work presents a novel method to produce radioactive nanoparticles in a single step. The direct synthesis is obtained by irradiation of aqueous solutions of the metal precursor using neutrons and gammas from a nuclear reactor.

### SUMMARY

This summary is provided to introduce in a simplified form concepts that are further described in the following detailed descriptions. This summary is not intended to identify key features or essential features of the claimed subject matter, nor is it to be construed as limiting the scope of the claimed subject matter.

In at least one embodiment, a method of producing radioactive nanoparticles includes: providing an aqueous solution including gold; and irradiating the aqueous solution thereby producing nanoparticles, wherein the nanoparticles include at least one radioactive isotope of gold.

In at least one embodiment, radioactive nanoparticles are provided. The radioactive nanoparticles are produced by irradiating an aqueous solution thereby producing nanoparticles that comprise at least one radioactive isotope. The aqueous solution includes a metal precursor, and the nanoparticles include at least one radioactive isotope. The nanoparticles may have use in treating cancer.

In at least one example, the aqueous solution includes an Au salt.

In at least one example, the aqueous solution includes HAuCl₄.3H₂O.

In at least one example, the aqueous solution includes a chloride of gold in a mixture including deionized water, polyvinylpyrrolidone (PVP), and 2-propanol.

In at least one example, the mixture is bubbled with nitrogen to remove oxygen.

In at least one example, the radioactive isotope of gold includes ¹⁹⁸Au and ¹⁹⁹Au.

In at least one example, irradiating the aqueous solution includes a radiation field that includes neutrons and gamma rays.

In at least one example, the radiation field is provided by a nuclear reactor.

In at least one example, the aqueous solution further includes silver, wherein the nanoparticles include bimetallic nanoparticles.

In at least one example, the aqueous solution includes gold chloride and silver nitrate.

In at least one example, the aqueous solution includes gold chloride and silver nitrate in a mixture of polyvinylpyrrolidone, and 2-propanol.

In at least one example, the mixture is bubbled with argon to remove oxygen.

In at least one embodiment, a method of producing radioactive bimetallic nanoparticles includes: providing an aqueous solution including gold and silver; and irradiating the aqueous solution thereby producing bimetallic nanoparticles, wherein the bimetallic nanoparticles include at least one radioactive isotope of gold and one radioactive isotope of silver.

In at least one embodiment, radioactive bimetallic nanoparticles are provided. The radioactive bimetallic nanoparticles are produced by providing an aqueous solution including gold and silver, and irradiating the aqueous solution thereby producing radioactive bimetallic nanoparticles. The bimetallic nanoparticles include at least one radioactive isotope of gold and one radioactive isotope of silver. The bimetallic nanoparticles may have use in cancer therapy. Uses may include imaging, drug delivery and treatment.

In at least one example, irradiating the aqueous solution includes irradiating the aqueous solution for a period of time selected to control particle size distribution of the nanoparticles.

In at least one example, the bimetallic nanoparticles include at least one of core-shell nanoparticles and alloyed nanoparticles.

### BRIEF DESCRIPTION OF THE DRAWINGS

The previous summary and the following detailed descriptions are to be read in view of the drawings, which illustrate particular exemplary embodiments and features as briefly described below. The summary and detailed descriptions, however, are not limited to only those embodiments and features explicitly illustrated.

FIG. 1A is a TEM micrograph of obtained radioactive gold nanoparticles that were irradiated at 200 kW for 0.5 min (sample 1) in the Missouri University of Science and Technology Nuclear Reactor (MSTR).

FIG. 1B is a TEM micrograph of obtained radioactive gold nanoparticles that were irradiated at 200 kW for 1 min (sample 2) in the MSTR.

FIG. 1C is a TEM micrograph of obtained radioactive gold nanoparticles that were irradiated at 200 kW for 3 min (sample 3) in the MSTR.

FIG. 1D is a TEM micrograph of obtained radioactive gold nanoparticles that were irradiated at 200 kW for 5 min (sample 4) in the MSTR.

FIG. 1E is a TEM micrograph of obtained radioactive gold nanoparticles that were irradiated at 200 kW for 10 min (sample 5) in the MSTR.

FIG. 1F is a TEM micrograph of obtained radioactive gold nanoparticles that were irradiated at 200 kW for 30 min (sample 6) in the MSTR.

FIG. 1G is a TEM micrograph of obtained radioactive gold nanoparticles that were irradiated at 200 kW for 60 min (sample 7) in the MSTR.

FIG. 2 is a plot of the variation of average particle size and standard deviation with the irradiation time.

FIG. 3 is a plot of particle population versus particle size in nm for sample 5.

FIG. 4 is an Energy Dispersive Spectroscopy (EDS) spectrum for sample 5.

FIG. 5A represents a method, in at least one embodiment, of producing radioactive gold nanoparticles by radiolysis of an aqueous gold solution, in a single step, using a nuclear reactor.

FIG. 5B shows the production of radioactive BMNPs by radiolysis of water, in a single step, using a nuclear reactor.

FIG. 6 illustrates, according to prior art, differences between core-shell clusters production and alloyed cluster production.

FIG. 7A is a micrograph of Au/Ag BMNPs obtained from the irradiation of a first specimen (1) (% Au/Ag 70/30) in the MSTR.

FIG. 7B is a micrograph of Au/Ag BMNPs obtained from the irradiation of a second specimen (2) (% Au/Ag 50/50) in the MSTR.

FIG. 7C is a micrograph of Au/Ag BMNPs obtained from the irradiation of a third specimen (3) (% Au/Ag 30/70) in the MSTR.

FIG. 7D is a micrograph of Au/Ag BMNPs obtained from the irradiation of a fourth specimen (4) (% Au/Ag 0/100) in the MSTR.

FIG. 8A is an EDS Spectrum of Au/Ag nanoparticles of the irradiated specimen 1.

FIG. 8B is an EDS Spectrum of Au/Ag nanoparticles of the irradiated specimen 2.

FIG. 8C is an EDS Spectrum of Au/Ag nanoparticles of the irradiated specimen 3.

FIG. 8D is an EDS Spectrum of Au/Ag nanoparticles of the irradiated specimen 4.

FIG. 9A is an SADP for pure gold in color-negative image format.

FIG. 9B is an SADP for pure silver in color-negative image format.

FIG. 10A is a TEM micrograph of a first specimen 1.

FIG. 10B is an SADP, in color-negative image format, of a core-shell nanoparticles obtained from specimen 1.

FIG. 11A is an indexed SADP, in color-negative image format, of an alloyed nanoparticle from specimen 3.

FIG. 11B is an SADP, in color-negative image format, of an alloyed nanoparticle from specimen 3 showing radii measurements.

### DETAILED DESCRIPTIONS

These descriptions are presented with sufficient details to provide an understanding of one or more particular embodiments of broader inventive subject matters. These descriptions expound upon and exemplify particular features of those particular embodiments without limiting the inventive subject matters to the explicitly described embodiments and features. Considerations in view of these descriptions will likely give rise to additional and similar embodiments and features without departing from the scope of the inventive subject matters. Although the term “step” may be expressly used or implied relating to features of processes or methods, no implication is made of any particular order or sequence among such expressed or implied steps unless an order or sequence is explicitly stated.

Any dimensions expressed or implied in the drawings and these descriptions are provided for exemplary purposes. Thus, not all embodiments within the scope of the drawings and these descriptions are made according to such exemplary dimensions. The drawings are not made necessarily to scale. Thus, not all embodiments within the scope of the drawings and these descriptions are made according to the apparent scale of the drawings with regard to relative dimensions in the drawings. However, for each drawing, at least one embodiment is made according to the apparent relative scale of the drawing.

At least one embodiment according to these descriptions includes a novel method to produce radioactive gold nanoparticles (AuNPs) in a single step. The synthesis is done in at least one example by radiolysis of aqueous solutions of gold chloride in a nuclear reactor, such as the Missouri University of Science and Technology Nuclear Reactor (MSTR). The resulting nanoparticles can be characterized using Transmission Electron Microscope (TEM) (Technai F20) and the Java-based image processing program ImageJ.

For example, a solution of gold chloride in a mixture of deionized water, polyvinylpyrrolidone (PVP), and 2-propanol can be prepared. The concentration ratio of 2-propanol/PVP can be 60. Seven different such samples of the solution were irradiated at reactor thermal power of 200 kW for 0.5, 1, 3, 5, 10, 30, and 60 min. The obtained nanoparticles have a size distribution between 3 nm and 450 nm. It is found that the particle size depends on the time of irradiation; longer irradiation time produces smaller nanoparticles. Some agglomerated particles are found in all samples. The resulting nanoparticles are characterized with Transmission Electron Microscopy (TEM) and ImageJ.

In at least one procedure, the chemical gold precursor is hydrogen tetrachloroaurate (III) trihydrate (HAuCl₄.3H₂O), ACS, 99.99% (metal basis), Au 49.5% min. The surfactant used (colloidal stabilizer) can be polyvinylpyrrolidone (PVP), 99.99%. These reagents are commercially available from chemical suppliers, including for example, Alfa-Aesar. The radical scavenger can be 2-propanol and the medium can be purified and deionized water.

A solution of 2 mM of HAuCl₄.3H₂O can be prepared using deionized water at room temperature. Dissolutions of PVP and 2-propanol can be added to the gold solution with a concentration ratio of 2-propanol/PVP=60. This ratio does not lead to any thermal reduction of the Au salt. The mixture solution can be bubbled with pure nitrogen for 30 min to remove oxygen.

The irradiation process, in some examples, was done in the MSTR operating at thermal power of 200 kW. Seven different samples, of 2 ml each, were irradiated for 0.5, 1, 3, 5, 10, 30 and 60 min. The morphology of nanoparticles was characterized using TEM (Technai F20) and the size distribution was analyzed using ImageJ.

Production of chemically stable radioactive AuNPs in a single step from the irradiation of the precursor solution is thus provided. In prior art approaches, nanoparticles were first synthesized by chemical or physical processes, and subsequently, the obtained nanoparticles were activated with a neutron source. In novel approaches according to these descriptions, irradiating the sample has the dual effects of producing nanoparticles and useful radioactive isotopes.

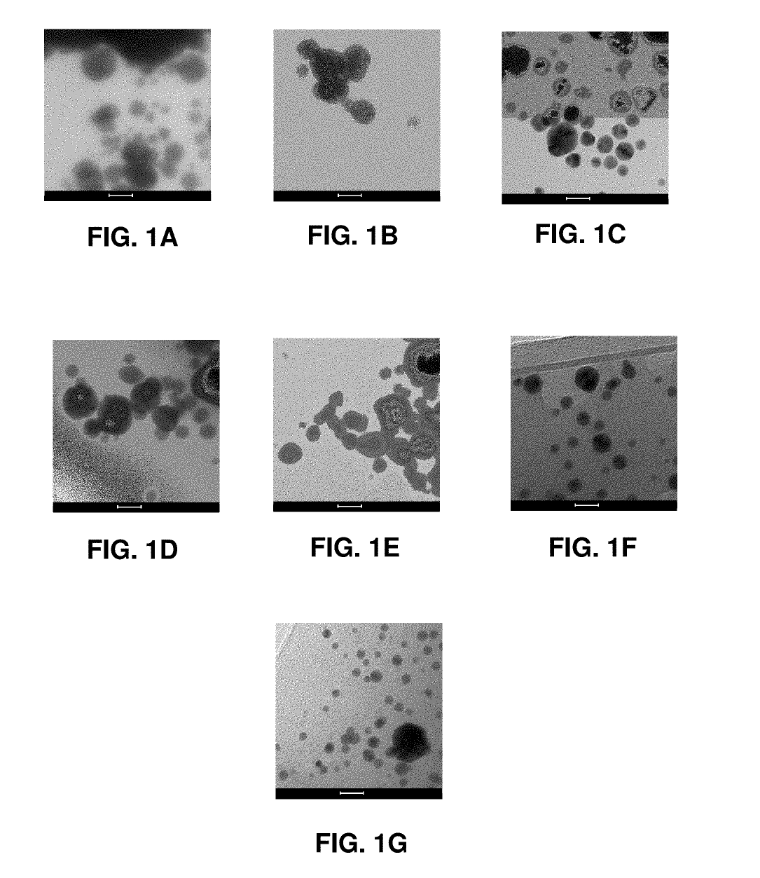

The synthesis of nanoparticles can be initiated by the radiolytic reduction of water by neutrons radiation and ionizing radiation. This reduction process creates solvated electrons (e_{aq}), H⁺, OH⁻, H₂O₂, and H₂ that are very reactive and reduced the metal ions present in the solution. After the irradiation process, the solution changes color from light yellow to dark reddish. Samples that were irradiated for a short time presented lighter reddish color than samples that were irradiated for longer time. The difference in color change is due to the particle size obtained with different irradiation durations. FIGS. 1A-1G shows TEM images of the seven samples, from shorter to longer irradiation time. The average particle size and their corresponding standard deviation (σ) are shown in Table 1, which lists average particle size (nm) and variation with time of irradiation.

TABLE 1

| | Irradiation Time | Average Size | σ | σ |
|---|---|---|---|---|
| Sample | (min) | (nm) | (nm) | (%) |
| 1 | 0.5 | 271 | 207 | 76 |
| 2 | 1 | 221 | 126 | 57 |
| 3 | 3 | 124 | 75 | 60 |
| 4 | 5 | 86 | 47 | 55 |
| 5 | 10 | 56 | 34 | 61 |
| 6 | 30 | 37 | 16 | 43 |
| 7 | 60 | 19 | 8 | 42 |

The TEM images show the presence of AuNPs in all samples, meaning that it is possible to synthesize NPs even with a short irradiation time. Despite of the addition of PVP to the solution, it may not be possible to avoid the agglomeration of particles. However, most of the particles remained non-agglomerated. This agglomeration may be a consequence of the elapsed time between the irradiation process and the TEM analysis. A delay provided time for the sample activity to decay enough according to safety regulations to be safely transported and analyzed with the microscope.

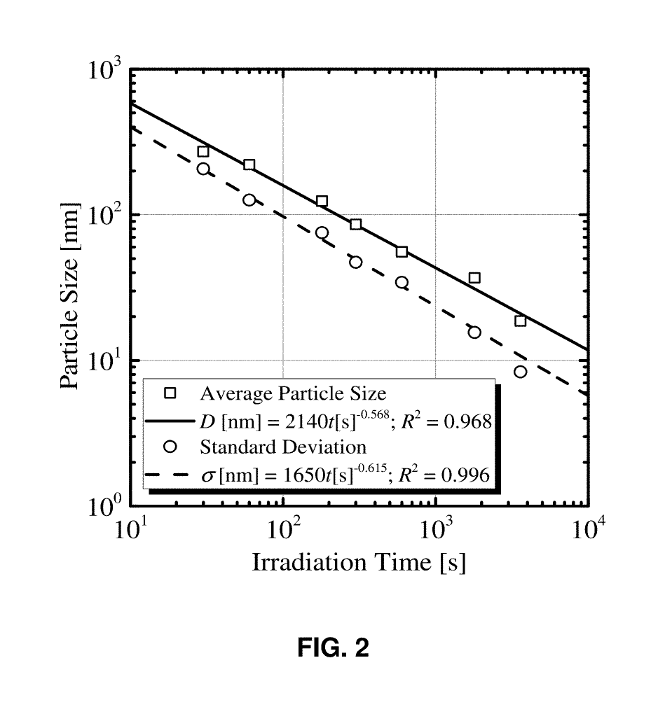

The variation of the average particle size and standard deviation with the irradiation time is shown in FIG. 2. Both the particle size and standard deviation of AuNPs decrease with longer radiation times following a power trend with R-squared values of 0.968 and 0.996, respectively. After sixty minutes of irradiation a particle size reduction of 93% was achieved. However, most of the particle size reduction happened during the first ten minutes of irradiation (79%). After that time, the reduction rate lowered down to 3% every ten minutes. The same behavior was observed for the standard deviation.

The size distribution of AuNPs used in cancer treatment is a key element to improve tumor retention, interstitial interaction inside of the body, and the cancer cell killing process. Particles with diameters between 1 nm and 100 nm are smaller than the size of the pores in the typical tumor vasculature allowing the AuNPs to access the cells in the tumor. However particles with diameters smaller than 10 nm can be removed from the body through the kidneys. Nanoparticles with approximately 50 nm of diameter have previously been shown to exhibit greater sensitization and higher cell uptake compared with other sizes.

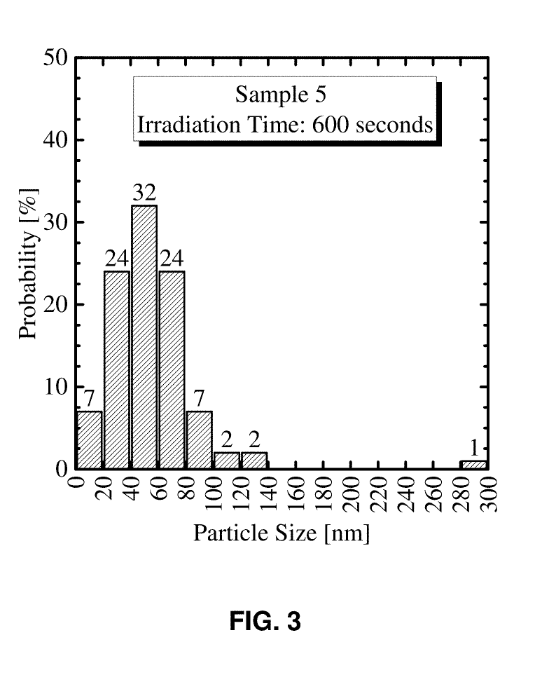

Thus, 50 nm may represent a promising particle size or diameter for cancer treatment. Advantageously, it is possible to obtain radioactive gold nanoparticles with appropriate characteristics to be used in cancer treatment following the synthesis conditions used with sample 5 (10 min of irradiation time at thermal power of 200 kW). The average particle size is 56 nm with a standard deviation of 34 nm. FIG. 3 shows the particle population vs. particle size for sample 5. Most of the obtained particles (95%) fall in the acceptable size range for cancer treatment (10 nm-100 nm) and almost half of them (32%) are the preferred particle size for cancer treatment (40 nm-60 nm). The biggest particle size obtained in sample 5 was 296 nm; probably from particle agglomeration since it falls in an isolated size range.

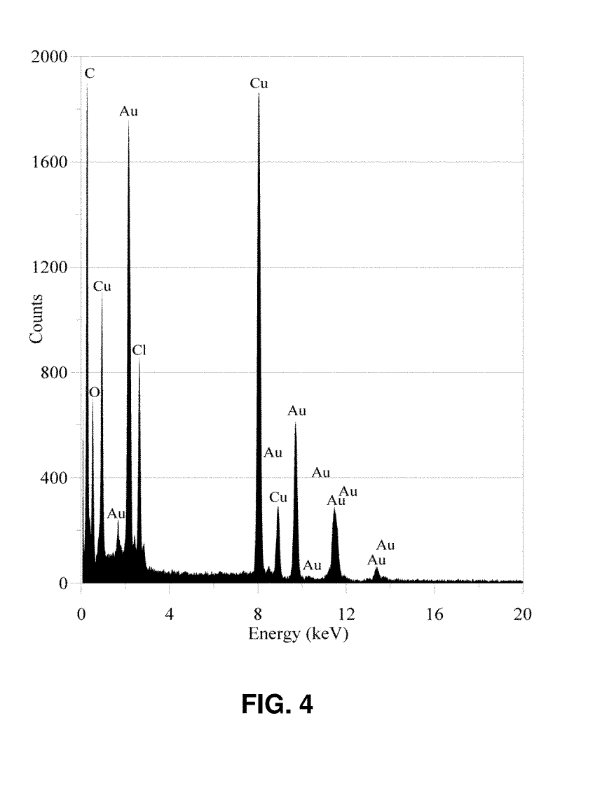

Chemical composition was verified using Energy Dispersive Spectroscopy (EDS) in order to determine the possible presence of other chemical species. FIG. 4 shows the EDS spectrum for sample 5 and Table 2 shows the composition in weight percentage and atomic percentage with the uncertainty for each element found in the spectrum. These analyses were performed for each sample and the obtained results are similar. The TEM sample holders used were Formvar/Carbon 300 mesh copper holders, the medium used to prepare the sample was deionized water and the samples only contained gold chloride as a principal precursor. For that reason, the Cu, and C presence in the spectrum was ignored.

TABLE 2

| element | weight % | atomic % | uncert. % |
|---|---|---|---|
| Cl (k) | 28.3 | 68.7 | 3.0 |
| Au (L) | 71.7 | 31.3 | 9.9 |

The weight percentage of gold in the sample demonstrates effective reduction of gold, producing metallic AuNPs without chemical contamination. The presence of Cl is expected due to the precursor. The Cu and C presence in the spectrum may be due to the sample holder.

Thus, the synthesis in a single step of radioactive AuNPs by water radiolysis is provided according to above-described examples and embodiments. The gold nanoparticles produced may present good characteristics for use in cancer treatment. In-vivo studies of radioactive gold nanoparticles to identify their suitability and effectiveness are within the scope of these descriptions.

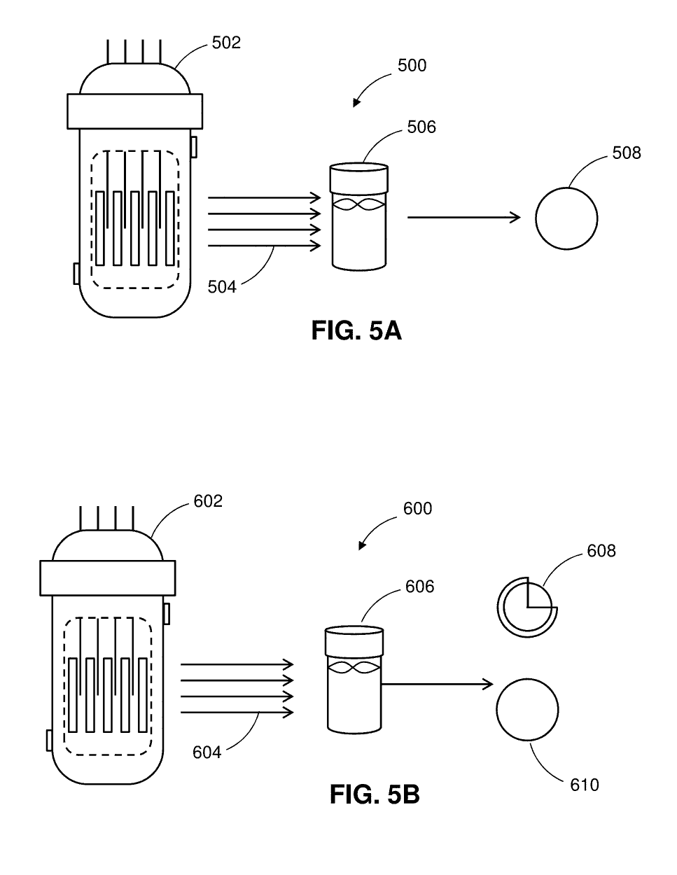

Accordingly, a method 500, in at least one embodiment, of producing radioactive gold nanoparticles by water radiolysis is shown in FIG. 5A. A nuclear reactor 502 provides a radiation field 504 including neutrons and gamma rays. An aqueous solution 506 containing gold is irradiated by the radiation field 504 and gold nanoparticles 508 are thereby produced. The aqueous solution 506 may include Au salt, for example HAuCl₄.3H₂O. The nanoparticle 508 shown in FIG. 5A may include multiple radioisotopes of gold produced, including for example, ¹⁹⁸Au and ¹⁹⁹Au, which are radioactive isotopes useful for locally irradiating and killing tumor cells.

In other embodiments, the synthesis of radioactive bimetallic nanoparticles by radiolysis can be conducted, for example using a nuclear reactor such as the Missouri S&T research nuclear reactor (MSTR). In at least one example, an aqueous solution of gold chloride and silver nitrate in a mixture of polyvinylpyrrolidone, and 2-propanol was prepared. The molar concentration ratio of 2-propanol/PVP was 60. Four different specimens with concentration ratio in percentage by volume Au to Ag of 70%/30%, 50%/50%, 30%/70%, and 100% of Ag were irradiated at a reactor thermal power of 10 kW for 3 minutes. The morphology, crystal structure, and chemical composition of the resulting nanoparticles where characterized using Transmission Electron Microscopy (TEM), Gatan Microscopy Suite Software and the Java-based image processing program ImageJ. The production of core-shell nanoparticles and alloyed nanoparticles was successfully accomplished and it was found that the obtained structure depends on the composition of the irradiated specimen.

FIG. 5B represents a method 600, according to at least one embodiment, of producing radioactive BMNPs by water radiolysis, in a single step, using a nuclear reactor. A nuclear reactor 602 provides a radiation field 604 including neutrons and gamma rays. An aqueous solution 606 containing gold and silver is irradiated by the radiation field 604 and bimetallic core-shell nanoparticles 608 and bimetallic alloyed nanoparticles 610 are thereby produced. The aqueous solution 606 may include, for example gold chloride and silver nitrate. Two species of produced nanoparticles 608 and 610 are shown in FIG. 5B to represent that multiple types of bimetallic nanoparticles may be produced, including for example, core-shell structure nanoparticles 608 and alloyed metal nanoparticles 610.

Water radiolysis can be represented by equation 1:

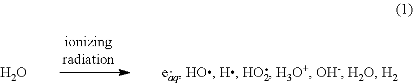

The species obtained from water radiolysis are highly reactive and can reduce metal ions. When a solution of metallic ions is irradiated, the reduction of metallic ions is reached until zero valence as shown in equations 2 and 3:

M⁺+e_{aq}⁻→M⁰ (2)

M⁺+H⁺→M⁰H⁺ (3)

After the reduction process, the atoms dimerize or associate with other ions forming metal clusters that produce metallic nanostructures by coalescence processes. The bimetallic nanoparticles can be obtained as an alloy of the metals or as a core-shell structure.

The production of core-shell clusters by radiolysis depends on the electro chemical characteristics of the metals ions in solution. When a metallic ion (Mₐ⁺) is in a solution with other metallic ion (M_{b}⁺) both of them have the same probabilities to be reduced by radiolytic radicals produced in water radiolysis. However, the less noble metallic ion acts as an electron donor to the nobler metallic ion and a further electron transfer between them occurs favoring first the reduction of the nobler metal. Assuming Mₐ⁺ as the nobler metallic ion and M_{b}⁺ as the less noble metallic ion, equation 4 shows the reaction of the reduction of the nobler metallic ion by electron transfer from the less noble atom.

Mₐ⁺+M_{b}⁰→(MₐM_{b})⁺→Mₐ⁰+M_{b}⁺ (4)

When the metallic ion is plurivalent the probability of segregation increases with the electron transfer between the intermediate-low valences of both metals. The nobler metallic ions receive electrons from the less noble metallic ions and monometallic clusters Mₐ are obtained first. Then, when the Mₐ⁺ ions are spent, the M_{b}⁺ ions are reduced thereafter on the surface of Mₐ. The final result is the production of a core-shell cluster where the nobler metal is coated by the lesser one.

The production of alloyed clusters occurs when the intermetal electron transfer is not obtained even during long irradiation times. The metal ions are reduced by the radicals obtained from water radiolysis (equation 1). This process produces atoms of both metals and it is followed by mixed coalescence. Equations 5 and 6 represent the coalescence process of the metallic atoms:

Mₐ+M_{b}→(MₐM_{b}) (5)

(MₐM_{b})+Mₐ→(M_{a,2}M_{b}) (6)

The union of atoms and clusters with ions is also possible during the reduction process and the reaction is represented in equation 7:

(MₐM_{b})⁺+Mₐ⁺→(M_{a,2}M_{b})²⁺ (7)

These associations allow the production of bimetallic alloyed clusters according to the relative initial ion concentration.

The formation of an alloyed or a core-shell cluster depends on the kinetic competition of the electron transfer from the less noble to the nobler metal ions (equation 4) and on the radiation-induced reduction of both metal ions (equation 7). This competition depends on the chemical characteristics of the metal and also on the dose rate.

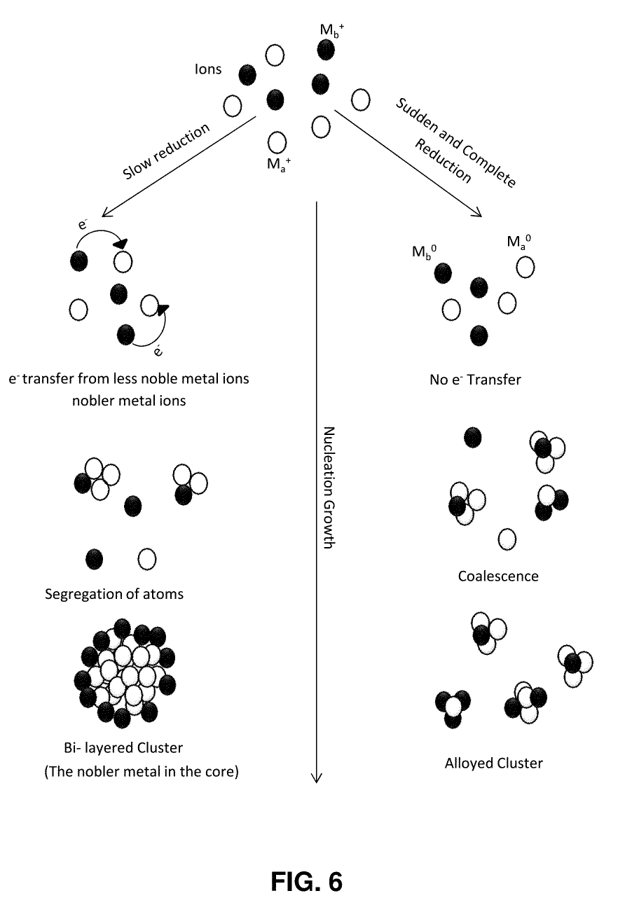

A powerful and sudden irradiation process could prevent the electron transfer between both metal ions, and the metallic atoms are produced fast and with total reduction. Then, the coalescence of those atoms forms alloyed clusters. In other words, a high dose rate of ionizing irradiation may kinetically favor alloyed metal nanoparticles rather than the normally formation of core-shell nanoparticles, which are favored if the formation reactions occur under thermodynamically controlled condition. FIG. 6 shows the difference between core-shell clusters production and alloyed cluster production.

As described herein, according to at least one embodiment, radioactive gold/silver BMNPs are produced by radiolysis of water, using a nuclear reactor, for example the MSTR. AuAg-BMNPs have good optical properties that depend on the composition, geometrical structure, and capability of adsorbing near infrared irradiation. Their applications include biomedical recognition processes, biosensing, drug delivery, photothermal therapy, and molecular imaging.

The chemical gold precursor used in at least one example is hydrogen tetrachloroaurate (III) trihydrate (HAuCl₄.3H₂O), ACS, 99.99% (metal basis), Au 49.5% min, the chemical silver precursor is silver nitrate (AgNO₃), ACS, 99.9% (metal basis), and the surfactant used (colloidal stabilizer) was polyvinylpyrrolidone (PVP), 99.99%. These reagents are commercially available from chemical suppliers, including for example. Alfa-Aesar. The radical scavenger was 2-propanol and the medium was purified and deionized water.

In at least one example, two solutions of 2 mM of HAuCl₄.3H₂O and AgNO₃ were prepared using deionized water at room temperature. Dissolutions of PVP and 2-propanol were added to the solutions with a ratio by molar concentration of 1:60, this ratio does not lead to significant thermal reduction of the metallic salts. The mixture solution was bubbled up with Argon for 30 minutes to remove oxygen. The deaeration process is important to prevent oxidation reactions caused by dissolved oxygen before and during the irradiation process, allowing the oxidation-reduction process exclusively from water radiolysis.

The irradiation process can be conducted in a nuclear reactor. In at least one example, the MSTR was used operating at 10 kW of thermal power. Four different specimens of 2 ml were irradiated for 3 minutes. The compositions of the specimens are shown in Table 3. The morphology, chemical composition and crystal structure of the nanoparticles were characterized using Transmission Electron Microscopy (TEM-Technai F20), Gatan Microscopy Suite Software and the Java-based image processing program ImageJ.

TABLE 3

Composition (vol %)

| Specimen | HAuCl4•3H2O | AgNO3 |
|---|---|---|
| 1 | 70 | 30 |
| 2 | 50 | 50 |
| 3 | 30 | 70 |
| 4 | 0 | 100 |

According to at least one embodiment, Au/Ag BMNPs are produced. The synthesis of nanoparticles can be initiated by the reduction process due to the products obtained from water radiolysis. In such an example, the irradiated Au/Ag solutions can present a color change from light yellow to light reddish. This is caused by the reduction of the metallic salts and depends on the concentration of AgNO₃ and HAuCl₄.3H₂O in solution; as the Au/Ag molar ratio increases, the intensity of the reddish color increases. The silver nitrate can play a special role on the behavior of gold atoms during growth, the silver assisted growth is slower and gold atoms grow a structure with no defect.

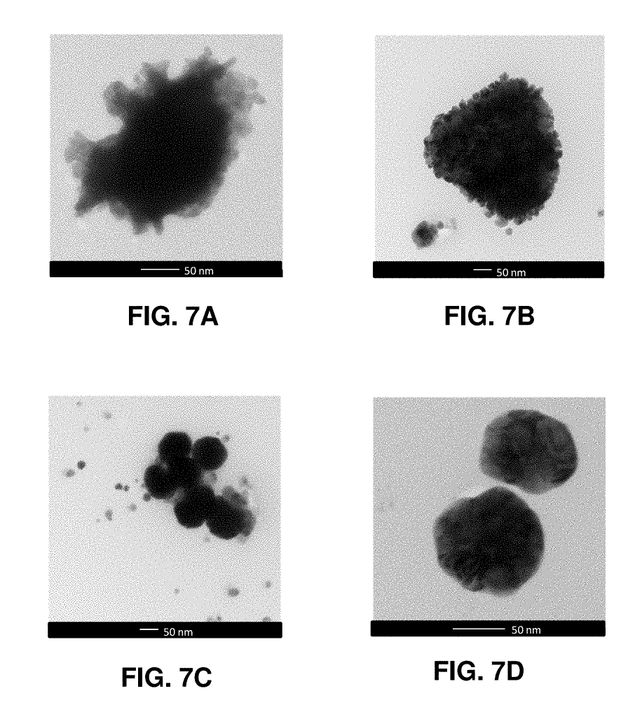

The morphology, crystal structure, and chemical composition analyses of the Au/Ag BMNPs show both core-shell and alloyed metal structures. FIGS. 7A-7D show the TEM images of the four specimens irradiated in the MSTR. The TEM micrographs show that particles from specimen 1 (FIG. 7A) presented a multi-spiked structure formed due to coating of metal nanoparticles. Particles from specimen 2 (FIG. 7B) presented a homogeneous deposition of metal nanoparticles on the surface. Specimens 3 (FIG. 7C) and 4 (FIG. 7D) presented nanoparticles with uniform surface without the deposition of nanoparticles. The results above are consistent with previous studies, demonstrating that specimens 1 and 2 presented nanoparticles with core-shell structure while specimen 3 presented nanoparticles with alloyed metal structure. Nanoparticles from specimen 4 are composed of pure Ag without the presence of other chemical components due to the composition of the irradiated specimen.

In order to confirm the bimetallic composition, the energy dispersive spectroscopy (EDS) and selected area diffraction patterns (SADPs) analysis were conducted to evaluate the chemical composition of the nanoparticles and their crystalline structure.

The EDS of specimen 1, 2 and 3 showed the presence of Au and Ag in each nanoparticle composition, proving that the production of BMNPs was achieved. However, the presence of Cl was also detected in all specimens; it could mean that the formation of AgCl occurred due to the chemical reaction between AgNO₃ and the Cl⁻ from the gold precursor. Therefore, the composition of the bimetallic nanoparticles could include some Au/AgCl. As mentioned before, specimen 4 presented only Ag in the nanoparticles composition because of the composition of the irradiated specimen.

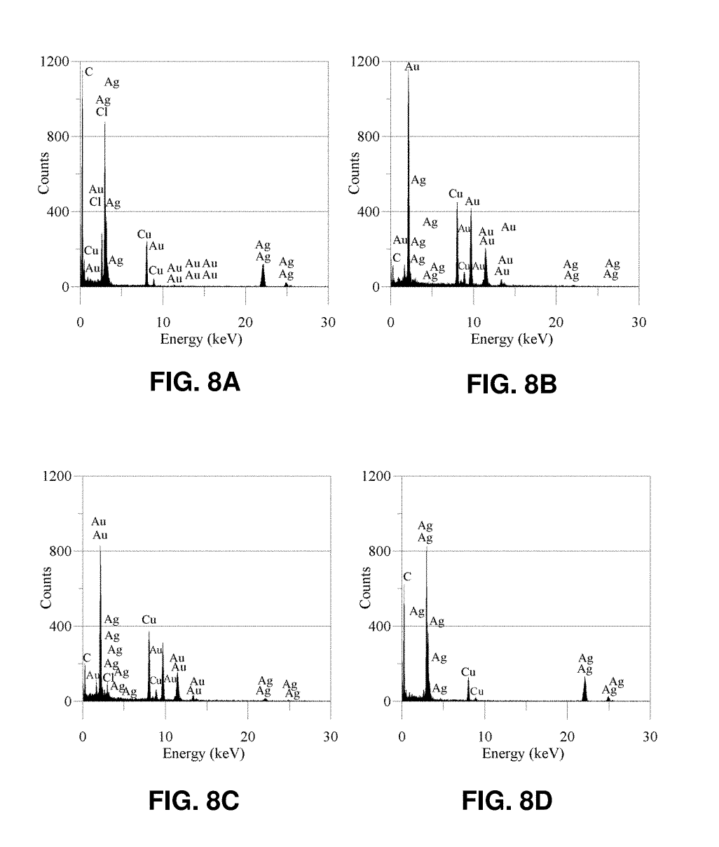

EDS spectra for the four analyzed specimens are presented in FIGS. 8A-8D. The composition in weight percentage, atomic percentage and uncertainty for each element found in the spectra is presented in Table 4. The presence of copper and carbon in the spectra is due to the composition of the TEM specimen holders (Formvar/Carbon 300 mesh).

TABLE 4

| | Weight % | Atomic % | Uncert. % | | Weight % | Atomic % | Uncert. % |
|---|---|---|---|---|---|---|---|
| Element | (± 0.01) | (± 0.01) | (± 0.01) | Element | (± 0.01) | (± 0.01) | (± 0.01) |
| Specimen 1 | | | | Specimen 2 | | | |
| Au | 10.88 | 10.41 | 0.19 | Au | 87.55 | 85.30 | 1.25 |
| Ag | 78.89 | 63.76 | 1.62 | Ag | 12.34 | 14.18 | 0.20 |
| Cl | 10.23 | 25.83 | 0.28 | Cl | 0.11 | 0.52 | 0.02 |
| Specimen 3 | | | | Specimen 4 | | | |
| Au | 70.21 | 61.74 | 1.32 | Au | 0.00 | 0.00 | 100.00 |
| Ag | 28.59 | 32.27 | 0.49 | Ag | 98.45 | 95.43 | 1.59 |
| Cl | 1.20 | 5.99 | 0.09 | Cl | 1.54 | 4.56 | 0.11 |

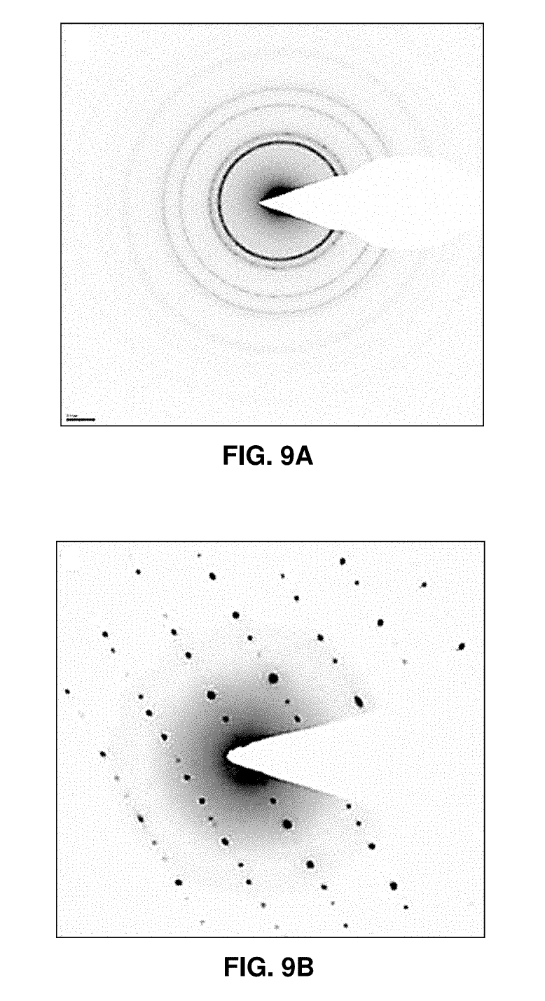

The SADPs of the Au/Ag BMNPs from the four specimens were analyzed comparing them to the SADPs of the pure metals (Au and Ag) and AgCl in order to verify the crystal structure and the presence of alloyed metal nanoparticles. Simulations of the Au, Ag, AgCl, and Au/Ag patterns were run using Web Electron Microscopy Applications Software (WebEMAPS) of the Department of Materials Science and Engineering, University of Illinois at Urbana-Champaign. The d-spaces for each pattern were obtained from the simulations. FIGS. 9A and 9B show the SADPs for pure gold (FIG. 9A) and pure silver (FIG. 9B). FIGS. 9A and 9B are shown in color-negative image format, relative to original images, for reproducibility. Table 5 shows the characteristics of the crystal structures of pure Au, pure Ag and AgCl.

TABLE 5

| | Au | Ag | AgCl |
|---|---|---|---|
| Atomic Radius | 174 pm | 165 pm | — |
| Crystal Structure | Face centered | Face centered | Face centered |
| | cubic | cubic | cubic |
| Lattice angles | π/2, π/2, π/2, | π/2, π/2, π/2, | π/2, π/2, π/2, |
| Lattice parameters | 4.078 | 4.085 | 5.549 |
| [Å] | | | |
| Space group name | Fm_3m | Fm_3m | Fm_3m |
| Space group number | 225 | 225 | 225 |

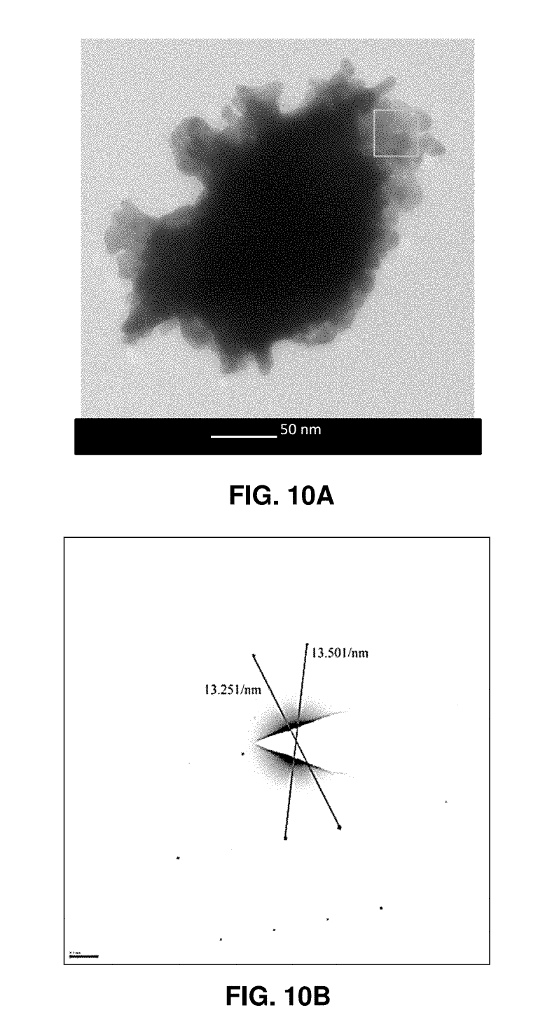

From Table 5, the crystal structures of the BMNPs are expected to be face centered cubic (FCC). According to the information presented in the introduction; the core-shell nanoparticles would present the SADP for pure silver (or AgCl) because the core is composed by Au atoms. The alloyed metal nanoparticles would present a crystal structure with lattice parameter between the values obtained for pure Au and pure Ag (or AgCl). Specimen 1, 2 and 4 presented similar SADP. Analyzing their patterns, it was found that the d-spaces obtained belong to the pure Ag with a lattice parameter of 4.087±0.001 Å, that means that the nanoparticles obtained from specimen 4 are pure Ag as are the shell structures of the nanoparticles obtained from specimen 1 and 2. The SADP obtained for specimen 1 (FIG. 10A) is shown in FIG. 10B, which is shown in color-negative image format, relative to an original image, for reproducibility.

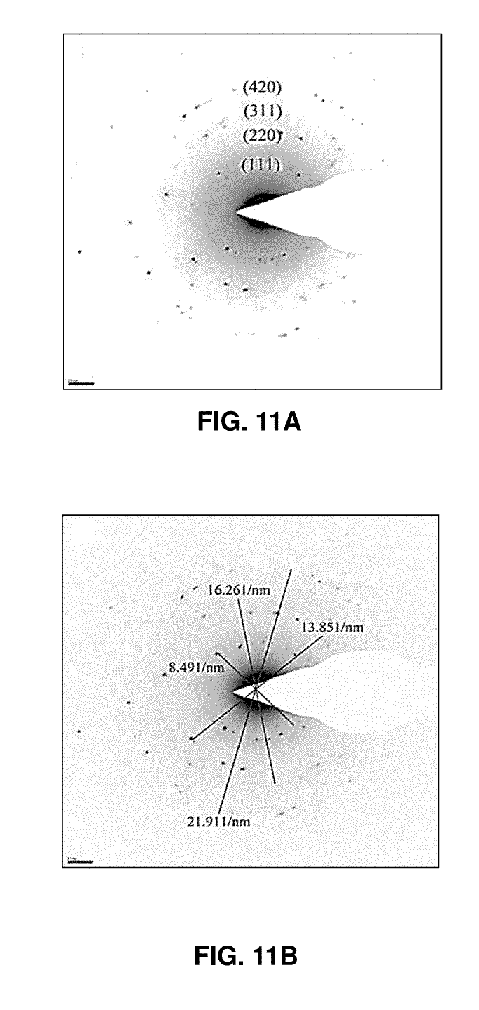

The SADPs, in color-negative image format relative to original image, are shown for specimen 3 in FIGS. 11A-11B. The diffraction pattern presents a ring arrangement, and the rings were indexed to confirm the crystal structure of the nanoparticles. The index process was done analytically and by running simulations using WebEMAPS. Also, the radii of the rings were measured to calculate the d-spaces of the crystals. FIG. 11A shows the indexed pattern and FIG. 11B shows the radii measurements of SADPs for BMNPs from specimen 3. Table 6 shows the radii (R) of the diffraction pattern, the ratio (Rn/R1), the obtained diffraction planes and d-spaces.

TABLE 6

| | Radius (R) | Ratio | Diffraction | d-space |
|---|---|---|---|---|
| Ring | [1/nm] | [Rn/R₁] | plane | [Å] |
| 1 | 4.245 ± 0.001 | 1.000 ± 0.001 | 111 | 2.361 ± 0.001 |
| 2 | 6.925 ± 0.001 | 1.631 ± 0.001 | 220 | 1.441 ± 0.001 |
| 3 | 8.130 ± 0.001 | 1.915 ± 0.001 | 311 | 1.230 ± 0.001 |
| 4 | 10.955 ± 0.001 | 2.580 ± 0.001 | 420 | 0.912 ± 0.001 |

The four rings in the SADP for the nanoparticles produced with the irradiation of specimen 3 correspond to the planes 111, 220, 311, and 420 reflections of the FCC crystal structure with lattice parameter of 4.082±0.001 Å. According to the data shown in Table 5, this lattice parameter is between the values for Au and Ag lattice parameters (4.078 Å and 4.085 Å respectively) which confirm the production of alloyed Au/Ag bimetallic structure. Since the lattice parameter values for Au and Ag are similar to each other, it is difficult to detect the random replacement of atoms of one element by atoms of another by using the electron diffraction analysis. However, it is possible to affirm that the production of alloy Au/Ag nanoparticles was successfully accomplished in specimen 3.

The presence of Cl⁻ in the EDS analysis is due to the chlorine in the gold precursor and the precipitation of AgCl. Additionally, from the SADP analysis it was confirmed that AgCl is not present on the surface of the BMNPs because the lattice parameter for the crystals obtained in specimen 3 (4.0816 Å) is smaller than the lattice parameter for AgCl (5.549 Å) and the precipitated particles of AgCl did not interact with the BMNPs.

Thus, in at least one example, the synthesis of Au/Ag bimetallic nanoparticles was successfully accomplished by water radiolysis for 3 minutes using the MSTR at 10 kW of reactor thermal power. Core-shell structures were obtained with the irradiation of solutions with Au/Ag concentrations of 70/30 vol % and 50/50 vol %, and it was found that the shell composition was pure Ag and the core composition was pure Au. Alloyed Au/Ag nanoparticles were obtained with the irradiation of a solution with Au/Ag concentration of 30/70 vol %.

It was confirmed that the alloyed metal nanoparticles presented an FCC crystal structure with a lattice parameter of 4.082±0.001 Å. The EDS analysis showed a composition of the alloyed metal of 70.21±0.01 wt % Au and 28.59±0.01 wt % Ag with uncertainty of 1.32±0.01% and 0.49±0.01% respectively. The presence of precipitated AgCl due to the chemical reactions between both precursors was also detected but this product was not found in the surface of the BMNPs, meaning that they did not act in the synthesis of the BMNPs.

Thus the synthesis of bimetallic Au/Ag nanoparticles is provided. Enhancing the production of the nanoparticles by including an inhibitor agent to avoid the precipitation of AgCl is within the scope of these descriptions. Additionally, the reactor power and irradiation time may each be varied in order to control the particle size distribution of the BMNPs.

Particular embodiments and features have been described with reference to the drawings. It is to be understood that these descriptions are not limited to any single embodiment or any particular set of features, and that similar embodiments and features may arise or modifications and additions may be made without departing from the scope of these descriptions and the spirit of the appended claims.

What is claimed is:

1. A method of producing radioactive nanoparticles in a single step, the method comprising:
providing an aqueous solution of a metal precursor; and
irradiating the aqueous solution thereby producing nanoparticles that comprise at least one radioactive isotope.

2. The method of claim 1, wherein the aqueous solution comprises Au salt.

3. The method of claim 2, wherein the aqueous solution comprises HAuCl₄ 3 H₂O.

4. The method of claim 1, wherein the aqueous solution comprises gold chloride in a mixture comprising deionized water, polyvinylpyrrolidone (PVP), and 2-propanol.

5. The method of claim 4, further comprising, before said irradiating, bubbling the mixture with an inert gas to remove oxygen.

6. The method of claim 1, wherein the radioactive isotope of comprises at least one of ¹⁹⁸Au and ¹⁹⁹Au.

7. The method of claim 1, wherein irradiating the aqueous solution comprises irradiating in a radiation field that includes neutrons and gamma rays.

8. The method of claim 7, wherein the radiation field is provided by a nuclear reactor.

9. The method of claim 1, wherein the aqueous solution further comprises silver, and the nanoparticles comprise bimetallic nanoparticles.

10. The method of claim 9, wherein the aqueous solution comprises gold chloride and silver nitrate.

11. The method of claim 9, wherein the aqueous solution comprises gold chloride and silver nitrate in a mixture of polyvinylpyrrolidone, and 2-propanol.

12. The method of claim 9, further comprising, before said irradiating, bubbling the mixture with an inert gas to remove oxygen.

13. The method of claim 1, wherein irradiating the aqueous solution comprises irradiating the aqueous solution for a period of time selected to control particle size distribution of the nanoparticles.

13. A method of producing radioactive bimetallic nanoparticles, the method comprising:
providing an aqueous solution comprising gold and silver; and
irradiating the aqueous solution thereby producing bimetallic nanoparticles,
wherein the bimetallic nanoparticles comprise at least one radioactive isotope of gold and one radioactive isotope of silver.

14. The method of claim 13, wherein the aqueous solution comprises gold chloride and silver nitrate.

15. The method of claim 13, wherein the aqueous solution comprises gold chloride and silver nitrate in a mixture of polyvinylpyrrolidone, and 2-propanol.

16. The method of claim 15, further comprising, before said irradiating, bubbling the mixture with an inert gas to remove oxygen.

17. The method of claim 13, wherein the radioactive isotope of gold comprises at least one of ¹⁹⁸Au and ¹⁹⁹Au.

18. The method of claim 13, wherein the bimetallic nanoparticles comprise at least one of: core-shell nanoparticles; and alloyed nanoparticles.

19. The method of claim 13, wherein irradiating the aqueous solution comprises irradiating in a radiation field that includes neutrons and gamma rays.

20. The method of claim 13, wherein the radiation field is provided by a nuclear reactor.
